(19) United States
(12) Patent Application Publication
(10) Pub. No.: US 2023/0348959 A1
(43) Pub. Date: Nov. 2, 2023

(54) REVERSE TRANSCRIPTION-FREE QUANTITATIVE-DISCRETE POLYMERASE CHAIN REACTION FOR POST-PROCESS QUALITY CONTROL OF VESICULAR BIOLOGICS

(71) Applicant: Wayne State University, Detroit, MI (US)

(72) Inventor: Thomas H. Linz, Livonia, MI (US)

(73) Assignee: Wayne State University, Detroit, MI (US)

(21) Appl. No.: 18/308,611

(22) Filed: Apr. 27, 2023

Related U.S. Application Data
(60) Provisional application No. 63/335,716, filed on Apr. 27, 2022.

(51) Int. Cl. C12Q 1/686; C12Q 1/6848

(52) U.S. Cl. CPC C12Q 1/6848; C12Q 1/686

(57) ABSTRACT

Methods discussed herein are directed to polymerase chain reaction (PCR) techniques, and more specifically quantitative-discrete PCR, wherein individual amplification reactions are performed on a per-payload basis among singly-captured and singly-isolated vesicles, for instance within individually sealed microwell reactors. These techniques enable post-process quality control measurements of per-vesicular manufacture loading efficiency and encapsulation efficiency of nucleic acid active ingredients in vesicular biologics, employing quantitative-discrete PCR techniques. By measuring variability of vesicular encapsulation of nucleic acids at a per-vesicular manufacture level of granularity, such techniques can enable collection of data that may be used to perform post-process formulation upon vesicular biologics, and to yield more homogenous formulations from both synthetic and biogenesis pathways. Furthermore, shortcomings of conventional PCR techniques which can introduce biases, false positives, and false negatives are eliminated.

### RELATED APPLICATIONS

This application claims priority to, and the benefit of U.S. Provisional Application No. 63/335,716 filed on Apr. 27, 2022, the contents of which are incorporated by reference in its entirety.

### INCORPORATION BY REFERENCE OF SEQUENCE LISTING

The contents of the text file named “W063-0092US Sequence Listing.xml,” which was created on Apr. 27, 2023 and is 6 KB in size, are hereby incorporated by reference in their entirety.

### BACKGROUND

In pharmaceuticals manufacturing, pharmaceutical formulation refers to the processing of drug ingredients into a pharmaceutical product. Pharmaceutical formulation requires, for example, that quantities of active ingredients of a drug be present in consistent quantities in a pharmaceutical product. In the absence of such consistency in formulation, dosages of pharmaceutical products could not be prescribed or administered with any degree of precision. Without precise dosages, drugs cannot be safely prescribed to patients.

Whereas active ingredients developed according to medicinal chemistry (i.e., small molecules) can be formulated in a substantially homogeneous fashion by various industrial processes, biologic active ingredients developed according to biopharmaceutical disciplines are substantially more complex and sensitive to various factors in formulation, as well as highly molecularly variable by nature. Thus, it is substantially more challenging to formulate biologics than small molecule drugs.

In a further challenge, various biologics must be packaged and delivered into target cells, as protection against enzymatic degradation. By way of example, nucleic acid-based drugs (such as, prominently, SARS-CoV-2 vaccines having a messenger RNA sequence as active ingredient, as well as gene-targeting therapeutics formulated using non-coding mimic and inhibitor RNA molecules), act by the delivery of nucleic acids into cells. This presents a non-trivial challenge, as nucleases degrade exogenous nucleic acids and cellular membranes prevent nucleic acids from entering cells.

Vesicles of various classes, being permeable to cellular membranes, are increasingly manufactured in biopharmaceutical processes as delivery vehicles for active ingredients of various biologics, to prevent them from absorption or degradation in biological environments. However, as manufacturing processes currently cannot formulate lipid nanoparticle drug products with substantially homogeneous concentrations of active ingredients, it is desirable to measure concentrations of active ingredients in lipid nanoparticle drug products, so as to achieve post-process quality control of the drug products and inform synthetic and biogenesis methods to increase homogeneity of subsequent formulations.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features. Some of the drawings submitted herewith may be better understood in color. Applicant considers the color versions of the drawings as part of the original submission and reserves the right to present color images of the drawings in later proceedings.

FIG. 1A illustrates capture of loaded vesicular manufactures onto an intermediate capture medium and the capture of an intermediate capture medium onto a visualization array according to example embodiments of the present disclosure.

FIG. 1B illustrates a visualization array according to example embodiments of the present disclosure.

FIG. 1C illustrates a logarithmic signal curve graphing a proportional relationship between digital signal (measured in percentage of fluorescing microwells out of all microwells of a plate) of imaged fluorescences against vesicular count expressed logarithmically according to example embodiments of the present disclosure.

FIG. 1D illustrates a signal curve graphing a sigmoidal relationship between a fluorescence intensity over time from each individual microwell and a number of polymerase chain reaction (PCR) thermal cycles according to example embodiments of the present disclosure.

FIG. 1E illustrates a fluorescence profile count aggregation according to example embodiments of the present disclosure.

FIG. 2A illustrates conjugating biotinylated DNA directly to streptavidin-coated beads.

FIG. 2B illustrates individual reactor environments according to example embodiments of the present disclosure being etched to include two different geometric forms.

FIG. 2C illustrates a Poisson distribution relationship between digital signal (measured in percentage of fluorescing microwells out of all microwells of a plate) of imaged fluorescences against nucleotide copies per bead expressed logarithmically.

FIG. 2D illustrates, for multiple calibration bead populations, respective aggregated fluorescence profile count histograms per reactor environment, to which curves are fitted illustrating threshold C_{q} arrivals.

FIG. 3A illustrates a series of fluorescence images showing one region of a multiwelled plate at increasing PCR cycle numbers.

FIG. 3B illustrates a fluorescence profile count aggregation histogram.

FIG. 4A illustrates a digital signal (measured in percentage of fluorescing microwells out of all microwells of a plate) of imaged fluorescences against FT-formed vesicular count expressed logarithmically according to example embodiments of the present disclosure.

FIG. 4B illustrates, over a liposome calibration range, respective aggregated fluorescence profile count histograms per reactor environment for FT-formed vesicular manufactures according to example embodiments of the present disclosure.

FIG. 5A illustrates a fluorescence profile count aggregation for loaded vesicular manufactures with variable DNA copy numbers according to example embodiments of the present disclosure.

FIG. 5B illustrates a reference fluorescence profile count aggregation for calibration beads overlaid on top of one vesicular population.

FIG. 6A illustrates a top-down SEM image of a region of a microwell array at 4,000x magnification.

FIG. 6B illustrates a tilted SEM image of an array device loaded with beads at 4,000x magnification. The dimensions of the microwells permit only a single bead to be loaded into each microwell.

FIG. 6C illustrates a top-down fluorescence image of a region of a microwell array after PCR at 8x magnification. Autofluorescence of beads is observed in every loaded microwell. Microwells originally containing a target miRNA also exhibit fluorescence in the signal acquisition region after PCR amplification.

FIG. 7A illustrates a side view (not to scale) of beads conjugated with DNA guides loaded into microwells;

FIG. 7B illustrates a side view (not to scale) of PCR master mix containing the target miRNA and primers introduced into the device.

FIG. 7C illustrates a side view (not to scale) of loaded microwells being sealed with oil to create individual reaction partitions.

FIG. 7D illustrates a side view (not to scale) of the PCR hot-start step releasing DNA guides into solution within the individually sealed microwells.

FIG. 8 illustrates digital signal of a quantitative-discrete PCR assay as described herein from positive controls (4 copies of let-7a per microwell) and negative controls (0 copies of let-7a per microwell) plotted against DNA guide numbers. 0 Copies is left bar of each pair; 4 copies is right bar of each pair.

FIG. 9 illustrates digital signal of a quantitative-discrete PCR assay as described herein plotted against PCR annealing temperature, using 300,000 guides/bead. Negative controls decreased in signal uniformly with increasing temperature, whereas positive controls experienced a sharp decrease at 60° C. 0 Copies is left bar of each pair; 4 copies is right bar of each pair.

FIGS. 10A and 10B illustrate digital signal of a quantitative-discrete PCR assay as described herein plotted against Cycle 1 annealing time. Long-start programs using annealing temperatures of 69° C. (FIG. 10A) and 65° C. (FIG. 10B) produced similar results between both positive and negative controls at each condition. Annealing for Cycles 2-35 was done for 20 s at the same annealing temperature as Cycle 1. 0 Copies is left bar of each pair; 4 copies is right bar of each pair.

FIG. 11 illustrates digital signal of a quantitative-discrete PCR assay as described herein plotted against Cycle 1 annealing temperature. Cold-start programs exhibited a gradual decrease in digital signal from positive controls as temperature increased, while negative controls decreased sharply between 46° C. and 53° C. An annealing temperature of 69° C. was used for Cycles 2-35. 0 Copies is left bar of each pair; 4 copies is right bar of each pair.

FIG. 12 illustrates digital signal of a quantitative-discrete PCR assay as described herein plotted against Cycle 1 annealing time and temperature. 0 Copies is left bar of each pair; 4 copies is right bar of each pair.

FIG. 13 illustrates analog fluorescence intensity signals of a quantitative-discrete PCR assay as described herein, for positive controls (dark grey) and negative controls (light grey) using uniform thermocycling (broken lines) with annealing at 58° C. for 20 s and asymmetric thermocycling (solid lines) with a 300-s long-start, in each case combined with a 53° C. cold-start for Cycle 1 and annealing at 69° C. for 20 s for Cycles 2-35. Each trace depicts the average fluorescence from all active wells at each thermocycle.

FIG. 14A illustrates digital signal (circles) and C_{q} values (triangles) of a quantitative-discrete PCR assay as described herein measured over a range of let-7a copy numbers in microfluidic microwell arrays. Concentration-dependent responses are observed in both the dPCR and qPCR dimensions.

FIG. 14B illustrates C_{q} values from bulk qPCR analyses over a range of let-7a copy numbers analyzed using a commercial qPCR instrument. A concentration-dependent response was not observed over this broad concentration range.

FIG. 15 illustrates averaged qPCR curves obtained from a commercial qPCR instrument. The copy numbers of let-7a did not affect the measured C_{q} values.

FIG. 16 illustrates aggregated fluorescence profile count histograms of quantification cycles of each of two populations of lipid nanoparticles, synthesized to contain either 3 copies or 24 copies of the miRNA miR-146a per lipid nanoparticle (LNP). LNPs loaded with 24 copies of miRNA (solid line) exhibit earlier C_{q} values and thus higher miRNA loading than LNPs loaded with 3 copies of miRNA (broken line).

FIG. 17A illustrates fluorescence of cell-derived vesicles transfected with the non-coding RNA let-7a, captured onto beads and then loaded into cylindrical microwells for discrete-quantitative PCR analysis.

FIG. 17B illustrates an aggregated fluorescence profile count histogram of the cell-derived vesicles of FIG. 17A.

### DETAILED DESCRIPTION

Methods discussed herein are directed to polymerase chain reaction (PCR) techniques, and more specifically quantitative-discrete PCR, wherein individual amplification reactions are performed on a per-payload basis among singly-captured and singly-isolated loaded vesicular manufactures, for instance within individually sealed microwell reactors.

Polymerase chain reaction (PCR) techniques include repeated thermal cycling of nucleic acid samples in a reactor environment wherein they are combined with reagents including at least a DNA polymerase and a nucleic acid primer. PCR reaction cycles are based on thermal cycles, which can be controlled by a temperature-controlling mechanical apparatus such as a thermal cycler. A thermal cycler can alternate between heating and cooling a reactor environment, causing nucleic acid strands in the reactor environment to, respectively, denature from respective complementary strands upon the reactor environment reaching a melting temperature to yield template strands, and, as template strands, anneal to primers upon the reactor environment reaching an annealing temperature.

The DNA polymerase, upon the reactor environment reaching an extension temperature (this being an activity temperature for the DNA polymerase), can act to elongate a primer annealed to a template strand by adding free nucleotides complementary to a template strand. (PCR techniques are commonly performed by combining nucleic acid samples with PCR “master mixes,” ready-made combinations of the above-mentioned reagents and other molecules at concentrations required for some number of reaction cycles.) A cycle of DNA polymerase activity yields nucleic acid strands complementary to template strands, substantially doubling the quantity of nucleic acid strands present in the reactor environment and yielding twice the number of template strands for further polymerase activity during a subsequent thermal cycle.

In the field of biopharmaceuticals, various classes of biologics may include nucleic acid molecules as active ingredients. By way of example, following the COVID-19 outbreak, RNA-based biopharmaceuticals were the first to gain FDA approval to begin vaccinating the general population. The active ingredient of these SARS-CoV-2 vaccines includes a messenger RNA (mRNA) sequence that is translated in vivo to produce a virus-specific protein, which stimulates an immune response.

Nucleotide-based biologic research is also ongoing into the therapeutic use of non-coding nucleic acids. Therapeutic uses of such non-coding nucleic acids include, by way of example, gene regulation pathways wherein binding of complementary sequences of target genes inhibits translation and gene expression.

The effectiveness of nucleic acid vaccines, non-coding nucleic acid therapeutics, and all nucleic acid-based biologics in general, hinges on the delivery of nucleic acid molecule payloads into cells. In a biological environment, natural enzymes such as nucleases degrade exogenous nucleic acids, and cellular membranes prevent nucleic acids from entering cells, making drug delivery significantly non-trivial.

In order to successfully deliver bioactive payloads into recipient cells while preventing enzymatic degradation, bioactive nucleic acids are commonly encapsulated within lipid nanoparticles in the process of manufacturing biologics. The successful development of mRNA COVID-19 vaccines has demonstrated efficacy of lipid vesicle-mediated drug delivery, and research continues in developing nucleotide-based biologics to treat numerous diseases.

Vesicles are generally microscale or nanoscale compositions wherein one lipid bilayer, or multiple concentric lipid bilayers, enclose an interior, which may be aqueous or composed of other substances. Vesicles can be manufactured by a variety of biogenesis pathways, giving rise to, most commonly, extracellular vesicles; or by synthetic processes, giving rise to liposomes (where both classes are further generalized as lipid nanoparticles). Biological vesicles can also be isolated from cell culture media or biofluid and then transfected with the nucleic acid drug. Utilizing naturally produced vesicles facilitates drug delivery by taking advantage of the membrane proteins and other biomolecules incorporated into the vesicle by its cell of origin. Biological vesicles may enhance targeted uptake of nucleic acid drugs by exploiting the biomolecules on the vesicle surface to trigger uptake into specific cells. The biopharmaceutical industry has developed a variety of drug products by applying various diverse biogenesis or synthetic manufacturing processes to cause vesicles of different classes to encapsulate quantities of various compositions, yielding microscale or nanoscale lipid-based vesicles which are biodegradable and biocompatible.

For the purpose of this disclosure, such manufacturing processes, whether biogenesis in nature or synthetic in nature or by any other pathway, shall be referred to as “manufacturing processes” or “processes,” and the products of such manufacturing processes, whether extracellular vesicular in nature, liposomal in nature, or of some other composition resulting in one or more lipid bilayers enclosing an interior, shall be referred to as “vesicular biologics” or “vesicular manufactures,” which may be loaded with some drug payload and referred to as “loaded vesicular manufactures.” By way of example, liposome formation techniques range from common methods such as ethanol injection and sonication to experimental methods such as the so-called supercritical carbon dioxide and dense gas methods.

Due to the inherent variability of biologics, it is understood in the biopharmaceutical industry that all manufacturing processes for vesicular drug products are subject to substantial variability. Unlike excipients and binders for small molecule pharmaceuticals which are typically inert, biomolecules are substantially more bioactive and exhibit high complexity, high specificity, and low stability, all presenting challenges in manufacturing processes. Thus, manufacturing processes across the biopharmaceutical industry commonly result in variability in their products.

Consequently, the formulation of consistent dosages requires post-process quality control. This, in turn, requires biologics manufacturers to inspect, measure, quantify, and assay vesicular manufactures by a variety of techniques. For example, quality control technicians can inspect vesicular size and characterize distributions of different sizes through techniques such as dynamic light scattering and nanoparticle tracking analysis. Quality control technicians can further simulate the release of loaded vesicular payloads in vitro, and can test the stability of vesicular manufactures.

Despite the use of such post-process quality control techniques, quality control technicians still must reckon with blind spots pertaining to loaded vesicular manufactures. Quality control technicians wish to quantify dosages of active ingredients loaded into vesicular manufactures during encapsulation processes, and, to this end, some quality control technicians have developed techniques to quantify so-called loading efficiency or encapsulation efficiency. However, even those quality control technicians who quantify loading efficiency or encapsulation efficiency of vesicular biologics have not conceived of techniques to quantify per-vesicular manufacture loading efficiency or per-vesicular manufacture encapsulation efficiency.

By merely lysing a population of vesicular manufactures, quality control technicians can run various assay and chromatography techniques upon the lysed suspension to derive average “loading efficiency” or “encapsulation efficiency” values based on the composition of the lysed suspension and the number of lysed vesicular manufactures of the population. However, while such averaged measurements may inform the design of manufacturing processes to some extent, they cannot be relied upon to determine formulation or dosages. This is because manufactured vesicular populations can exhibit substantial heterogeneity: various vesicular manufacturing processes, at scale, can yield individual vesicular manufactures which capture substantially variable concentrations of active ingredients. Consequently, mere random sampling from a vesicular population can yield substantially different dosages across different samples, which may not be predictable from sample quantity and may not be predictable from vesicular size.

Loading efficiency and encapsulation efficiency measurement techniques do not extend to per-vesicular manufacture loading efficiency and per-vesicular manufacture encapsulation efficiency. This inability to measure vesicular population heterogeneity leaves quality control technicians unable to ascertain number of nucleic acid copies delivered to cells by manufactured vesicular biologics.

Vesicular biologics are manufactured to encapsulate a particular target quantity of a payload of an active ingredient for delivery. With regard to nucleic acid active ingredients, for example, a target quantity of the nucleic acid active ingredient can be expressed as a particular copy number. Due to heterogeneity in manufactured vesicular populations, any sample vesicular population can include underloaded vesicular manufactures relative to the target quantity of the active ingredient (“underloaded vesicular manufactures”), and overloaded vesicular manufactures relative to the target quantity of the active ingredient (“overloaded vesicular manufactures”). Administering a vesicular biologic including substantial numbers of underloaded vesicular manufactures in vivo can render the drug ineffective, while administering a vesicular biologic including substantial numbers of overloaded vesicular manufactures in vivo can overdose cells and cause potentially harmful side effects.

Therefore, example embodiments of the present disclosure provide post-process quality control measurements of per-vesicular manufacture loading efficiency and encapsulation efficiency in vesicular biologics, employing quantitative-discrete PCR techniques. By measuring variability of vesicular encapsulation of nucleotides at a per-vesicular manufacture level of granularity, such techniques can enable collection of data that may be used (for instance, by formulation engineers and formulation technicians) to perform post-process formulation upon vesicular biologics, to work toward substantial homogeneity.

For the purpose of example embodiments of the present disclosure, an aqueous solution of nucleic acid molecules is encapsulated in some number of vesicular manufactures by any known vesicular formation technique. For the purpose of performing these vesicular formation techniques, vesicular manufactures are formed based on a target quantity of the nucleic acid active ingredient, expressed as a particular copy number (for example, 10 copies of the target nucleic acid per vesicular manufacture), and a target vesicular manufacture volume (a volume of aqueous solution to be encapsulated in each vesicular manufacture), from which a target nucleotide density is derived (by dividing the target copy number by the target vesicular volume). Based on the target nucleic acid density, the target vesicular volume, and a number of nucleic acid molecules to be dissolved in aqueous solvent for encapsulation, a target nucleotide concentration in solvent can be derived.

Nucleic acid molecules according to example embodiments of the present disclosure can include coding nucleic acids. By way of example, coding nucleic acids include DNA and messenger RNA (“mRNA”) molecules.

Nucleic acid molecules according to example embodiments of the present disclosure can include non-coding nucleic acids. By way of example, non-coding nucleic acids include microRNA (“miRNA”) molecules, small interfering RNA (“siRNA”) molecules, PIWI-interacting RNA (“piRNA”) molecules, small nuclear RNA (“snRNA”) molecules, small nucleolar RNA (“snoRNA”) molecules, and the like.

Additional types of RNA include repeat associated siRNA (rasiRNA), trans-acting siRNA (tasiRNA), CRISPR RNA (crRNA), transfer RNA (tRNA), Promoter-associated RNA (PASR), Transcription stop site associated RNAs, signal recognition particle RNA, transfer-messenger RNA (tmRNA), SmyRNA, small Cajal Body-specific RNA (scaRNA), Guide RNA (gRNA), Spliced leader RNA, ribosomal RNA (rRNA), Telomerase RNA, Ribonuclease P, or retrotransposons, satellite RNA, virioids, viral genomes or fragments thereof.

As a preliminary step, a thin-film technique was applied to form a lipid film for the subsequent operation of example liposome formation techniques. Solutes including 1.4 molar equivalents of POPC, 1 molar equivalent of cholesterol, and 0.014 molar equivalents of DSPE-PEG2000-biotin were dissolved in 0.5 mL chloroform. The resulting solutions were then dehydrated using, for instance, a rotary evaporator to remove solvent and form a thin film of lipid.

Starting from a lipid film formed such as described above, liposomes were formed by a freeze-thaw (“FT”) technique. A vial containing the lipid thin film and a tube containing 125 µL of non-biotinylated DNA (i.e., the target nucleic acid payload) were both heated in a water bath at 55° C. for 30 min. This target nucleic acid concentration (as referenced above) is derived from a target nucleotide density, the number of nucleotide molecules dissolved in solvent, and the target liposome volume of the 100 nm diameter liposomes to be formed (i.e., 520 zL).

The warmed nucleotide solution was added to the lipid vial and incubated in the water bath at 55° C. for 1 h stirring constantly. The incubated vials were placed in a -20° C. freezer until frozen (ca. 10 min) and then back into the 55° C. water bath to thaw (ca. 5 min). This freeze-thaw process was repeated four additional times to entrap DNA in the lipid vesicles.

Alternatively, starting from a lipid film formed such as described above, liposomes can be formed by an ethanol addition (“EA”) technique. 83 µL ethanol was added to a lipid thin film vial to dissolve the lipids. Non-biotinylated DNA (i.e., the target nucleic acid payload) (125 µL) in 10 mM tris HCl, pH 7.5 was heated in a water bath at 55° C. for 30 min. The lipid-ethanol solution was added dropwise to the nucleotide solution, causing liposomes to form and entrap the DNA within.

In each case, liposomes were extruded to a target diameter of 100 nm, for instance by first passing each sample through a 200 nm membrane 15 times using a Polaris extruder (Avanti Polar Lipids), then passed through a 100 nm membrane 20 times in a similar fashion. Theoretically, this yields liposomes with diameters of 100 nm, but it should be understood that resultant diameters are variable in practice (exhibiting a size distribution), as described subsequently. The extruded liposomes were dialyzed in dialysis tubing with a 1000 kDa molecular weight cutoff (Spectrum Laboratories, New Brunswick, NJ) in PBS/ 0.1% Tween for 24 h to remove unencapsulated DNA and excess lipid. The dialyzed liposomes were stored in dialysis buffer at 4° C. for up to 1 week.

Subsequently, the size distribution and particle concentrations of the formed liposome populations can be measured with a Spectradyne nCS1™ nanoparticle analyzer (Signal Hill, CA). The formed liposomes can also be imaged with a transmission electron microscope (“TEM”) (JEOL, Peabody, MA) after staining with 2% uranyl acetate to obtain liposome size and visualize morphology. Images obtained by TEM can be reviewed to verify liposome formation and preliminary particle sizes. Quantitative measurements of liposome sizes and concentrations can be made using a nanoparticle analyzer.

By way of example, liposome diameters of a formed liposome population as described below ranged between 65 and 150 nm, with 70 nm being the most prevalent diameter. Even given a target diameter of 100 nm for liposomes, such size variations within a formed liposome population falls within desired size ranges for pharmaceutical drug delivery. A target vesicular diameter and/or a target vesicular volume can be among specifications of vesicular and/or liposome manufacturing processes as described herein.

Additionally, upper ranges of liposome diameters reached as high as 700 nm, which suggests the formation of liposome aggregates. These larger liposomes are much higher in volume and thus likely contain substantially higher DNA copy numbers.

However, variable liposome diameters further impact variability of per-vesicular manufacture loading efficiency. For example, assuming 100-nm diameter liposomes are intended to contain 10 DNA copies: for 70 and 150-nm diameter liposomes, theoretical copy numbers loaded within are 3 and 34 DNA copies, respectively, based on the 10-fold difference in liposome volume, which may have biological consequences. Such extent of copy number heterogeneity is also expected to broaden measured threshold quantification cycle (“C_{q}”) arrival (as described below) distribution as detected by techniques described herein by >3 cycles, or even much more given 700-nm diameter liposomes.

Generally, for the purpose of example embodiments of the present disclosure, it should be understood that vesicular populations according to the target copy number, target vesicular diameter, target vesicular volume, target nucleic acid concentration, and such specifications as provided above, or according to any other similar or different specifications, are not limited to formation by the above-given techniques, and can be formed by any other suitable techniques, such as vesicles by various biogenesis pathways; liposomes by sonication, the supercritical carbon dioxide method, the dense gas method, and the like; and other techniques that will be recognized by those of skill in the art or equivalents subsequently developed. For additional information on vesicle types, see Yetisgin et al., Molecules 2020, 25, 2193, which describes in detail liposomes, solid lipid particles, and exosomes. For example, a solid lipid particle (SLP) refers to lipid components wherein at least one lipid component is solid at temperatures of at least 50° C. SLPs can also be defined including a lipid matrix that is solid at room and body temperatures that is stabilized by the presence of a surfactant.

Given a quantity of such formed vesicular populations, without limitation as to formation technique, example embodiments of the present disclosure provide a quantitative-discrete PCR technique which provides measurements of per-vesicular manufacture nucleotide loading, so as to determine whether actual nucleotide loading accords with a target copy number as specified above.

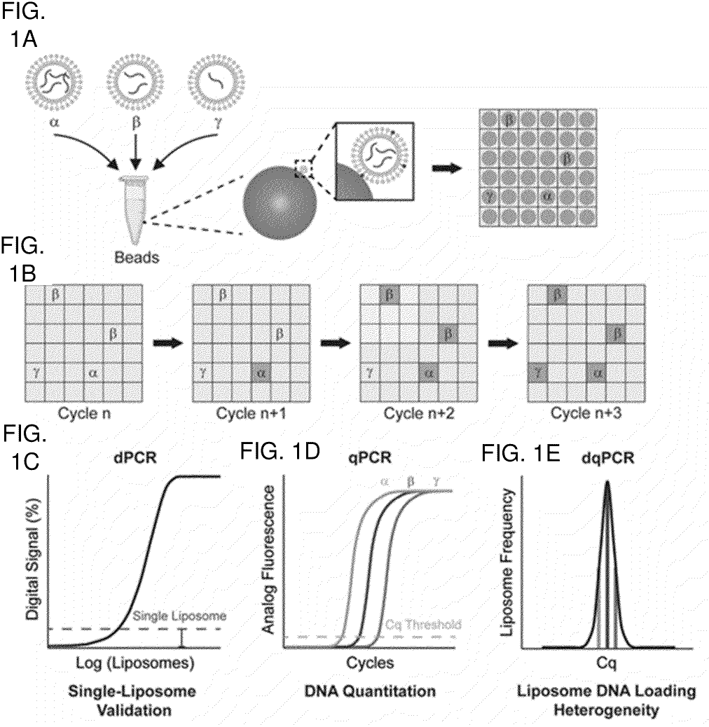

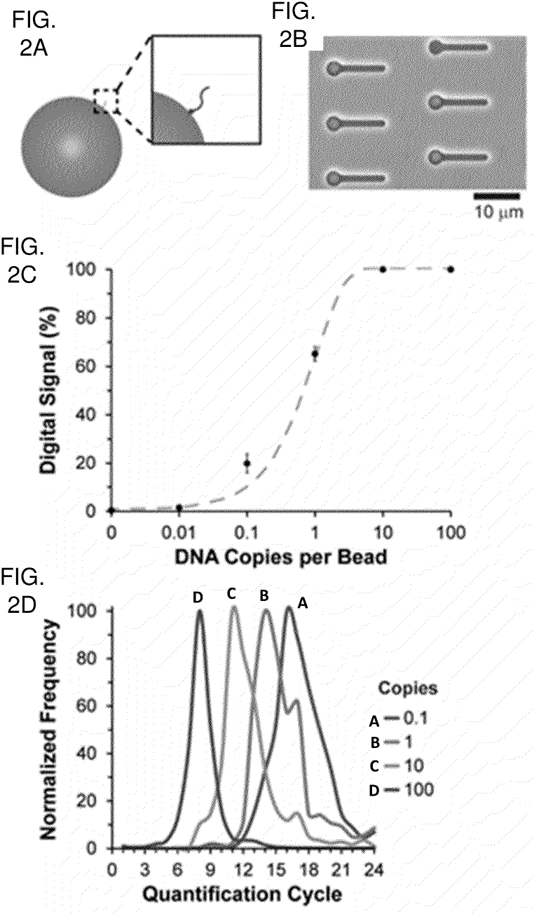

FIGS. 1A through 1E illustrate steps of a quantitative-discrete PCR technique according to example embodiments of the present disclosure.

FIG. 1A illustrates capture of vesicular manufactures onto an intermediate capture medium. According to example embodiments of the present disclosure, an intermediate capture medium is a physical medium effective to both capture vesicular manufactures, and to, itself, be captured within a reactor environment as shall be described subsequently. For example, an intermediate capture medium as illustrated in FIG. 1A can be a bioreceptor-bound magnetic bead.

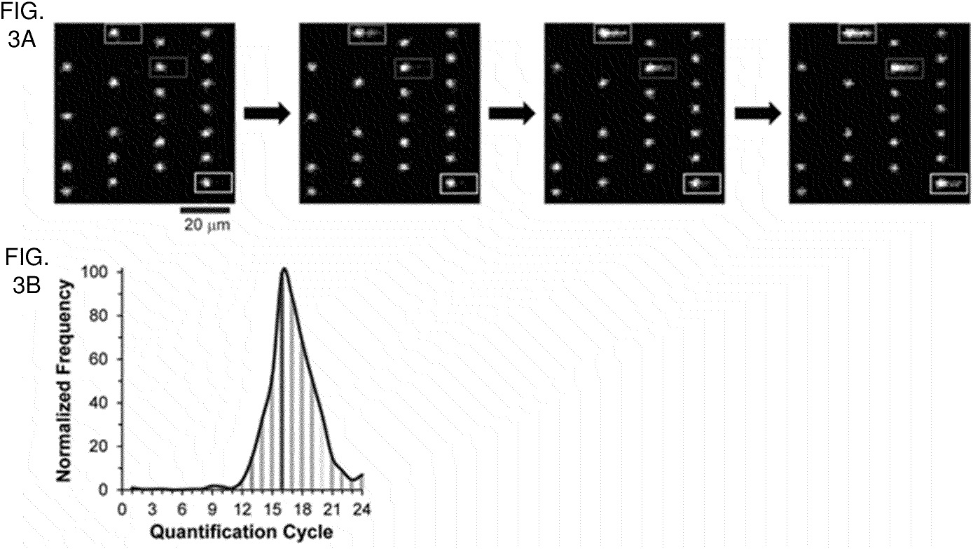

To prepare calibration beads (FIG. 2A), a streptavidin magnetic bead population was established by conjugating biotinylated DNA to streptavidin-coated beads (2.8 µm diameter). 6.5 million magnetic beads were aliquoted into a microcentrifuge tube and washed with 1x TBS/ 0.1% Tween 20. The washed beads were then incubated with 100 µL of biotinylated nucleic acids (i.e., a bioreceptor which binds biotinylated nucleic acids) (1 fM -10 pM) in TBS/0.1% Tween for 1 h on a tilt rotator. A nucleic acid concentration in the incubation is calculated to provide the desired conjugation density based on the numbers of bioreceptor molecules and beads in the incubation mixture, such that comprehensive binding between the biotin and streptavidin is theoretically achieved. The incubated bioreceptor-bound beads were then washed in TBS/ 0.1% Tween 20/ 0.1% BSA to remove residual DNA and passivate the bead surface. The prepared calibration beads were stored at 4° C.

It should be understood that any bioreceptors with strong affinity can be bound to the magnetic beads, such as antibodies, glutathione, maltose-binding protein, and the like.

The bioreceptor-bound magnetic beads perform as an intermediate capture medium according to example embodiments of the present disclosure. The streptavidin coating of the beads binds bioreceptors receptive to liposomes of a formed liposome population which is to undergo the quantitative-discrete PCR process. The beads themselves can, furthermore, be captured individually within reactor environments, as shall be subsequently described.

It should be understood that, for the purpose of example embodiments of the present disclosure, intermediate capture mediums can each capture zero or any non-zero number of vesicular manufactures upon incubation with a formed vesicular manufacture population. It is possible for one intermediate capture medium to bind more than one vesicular manufacture in the event that formed vesicular manufacture populations are high in concentration, though, at sufficiently low concentrations, intermediate capture mediums are highly likely to capture either zero or one vesicular manufacture. However, by operation of the quantitative-discrete PCR technique, quality control technicians can discern fluorescence from beads that have bound only one vesicular manufacture, and can verify that beads have captured only one, rather than more than one, vesicular manufacture. Therefore, techniques as described herein allow quality control technicians to derive discrete per-vesicular manufacture loading measurements without conflation with loading measurements taken from more than one vesicular manufacture. Such quantization to obtain discrete per-vesicular manufacture loading measurements shall be described in further detail subsequently.

FIG. 1B illustrates a visualization array according to example embodiments of the present disclosure. The visualization array includes an array of sealable reactor environments, each effective to capture no more than one intermediate capture medium as described above with reference to FIG. 1A.

By way of example, a visualization array as illustrated in FIG. 1B can be a multiwelled plate etched with a pattern of microwells. Silicon wafers (University Wafer, South Boston, MA) were primed with HMDS and then spin-coated with SPR 220 3.0 photoresist. Photolithography was performed to pattern microwell features onto the wafer using a custom photomask (e.g., Photronics, Brookfield, CT). Developed wafers were etched in a deep reactive ion etcher (SPTS Technologies, Milpitas, CA) to produce recessed microwells in the silicon. Photoresist was then stripped using AZ 726 developer followed by oxygen plasma cleaning (Yield Engineering Systems, Fremont, CA).

Each individual microwell according to example embodiments of the present disclosure can be etched to include two adjoined subwells of different geometric forms: a capture subwell and a reactor subwell. A capture subwell has a first geometric form sized to accept entrance of no more than one intermediate capture medium as described above, and a reactor subwell has a second geometric form sized to deny entrance of any intermediate capture medium as described above. As illustrated in FIG. 2B, the capture subwell has a substantially spherical form, and the reactor subwell has a substantially elongated form narrower in width than a diameter of the capture subwell.

By way of example, the capture subwell can be 3 µm in diameter, and the reactor subwell can be 1.5×10 µm in width and length, respectively. These dimensions can be verified with a scanning electron microscope (“SEM”) (JEOL).

Furthermore, the capture subwell and the reactor subwell can be substantially similar in depth, such as, by way of example, 4 µm in depth. A subwell depth can be verified using a profilometer (Bruker, Billerica, MA).

However, it should be understood that each individual microwell according to example embodiments of the present disclosure need not include any subwells. Rather, each individual microwell can include one geometric form without including a second geometric form.

A total of 32 devices, where each device constitutes a single multiwelled plate, were produced on one wafer with each device containing 25,000 microwells. Fluidic domes were then created by spinning SPR 220 3.0 photoresist onto B270 glass (Howard Glass, Worcester, MA). Dome features were patterned by exposing a second photomask and developing the photoresist. The substrate was then immersed in buffered oxide etchant to etch features to a depth of 15 µm. Entry ports were powder blasted into the glass (Comco Inc, Burbank, CA). Glass and silicon substrates were then silanized with trichloro(octyl) silane and epoxied together to form the final devices.

Thus, each multiwelled plate as illustrated in FIG. 1B includes many individual microwells each made up of two adjoined subwells. Each microwell can receive no more than one intermediate capture medium as described above in the capture subwell, while leaving sufficient room in the reactor subwell to allow PCR reactions to proceed, as described subsequently.

According to example embodiments of the present disclosure, formed liposome populations can be captured onto an intermediate capture medium, and the intermediate capture medium can then be captured on a visualization array. By way of example, a population of liposomes was manufactured via the FT method to contain 10 DNA copies/liposome with a target diameter of 100 nm; substantially most liposomes are expected to produce an “active” digital signal (as described below) resulting from this comparatively high DNA loading. Biotinylated lipids were included in the synthesis to provide receptor sites allowing intermediate capture mediums to capture liposomes by binding with biotinylated lipids on streptavidinylated beads. A formed liposome population was then incubated with a prepared bead population together for 1 h in PBS/ 0.1% Tween 20.

The bead population was then washed 3x with capture buffer and loaded into one or more multiwelled plate, individual beads being pulled into individual microwells by proximity of any magnetized object. By the geometric forms of the capture microwells, no more than one individual bead will be pulled into any individual microwell. This is demonstrated by illustration in FIG. 2B, a SEM image of six individual microwells, wherein five microwells contain captured beads.

PCR master mix was flowed into each multiwelled plate and allowed to fill each microwell, and each microwell was then sealed by emulsion by applying an oil coating over the multiwelled plate.

PCR was conducted on an AZ100 epifluorescence microscope (Nikon Instruments, Melville, NY) where the microscope stage is mechanically converted to electrically function as a thermal cycler apparatus. A two-step PCR reaction was induced for amplification, wherein melting and annealing/extension temperatures are 91° C. and 65° C. for 6 s and 20 s, respectively.

After every PCR thermocycling step, each microwelled plate was imaged with an Andor Zyla sCMOS camera (Oxford Instruments, Abingdon, UK) and a Sola SE light source (Lumencor, Beaverton, OR). Resulting images were input into a computing system running the FIJI image processing software package to identify individual fluorescences captured corresponding to each fluorescing bead imaged in the microwelled plate, thereby quantifying fluorescence intensity from each individual microwell as a signal over time updated after each PCR thermal cycle.

On a one-time basis after a final PCR amplification (or after any PCR amplification of the overall PCR thermocycling steps), endpoint fluorescence measurements were obtained from each individual reactor environment (i.e., each microwell). Reactor environments originally containing DNA exhibit high fluorescence and can be denoted as “active”. Reactor environments not containing DNA do not fluoresce and can be denoted as “inactive”. Samples with higher DNA copy numbers have higher digital signal (i.e., percentage of active partitions) than those with fewer copies.

FIG. 1C illustrates a logarithmic signal curve graphing a proportional relationship between digital signal (measured in percentage of fluorescing microwells out of all microwells of a plate) of imaged fluorescences against liposome count expressed logarithmically. A horizontal broken line illustrates a single-vesicular manufacture threshold: digital signals below this digital signal value threshold indicate a 95% likelihood of a single vesicular manufacture. Conversely, digital signals above this digital signal value threshold indicate <95% probability of single vesicular manufactures contained in each active well.

It should be understood that the particular digital signal value threshold represented by the horizontal broken line can vary depending on particular values of specifications of liposome manufacturing processes.

The principle of a digital signal value threshold as described above can be illustrated by an analogous relationship between digital signal value threshold and nucleic acids captured directly to a streptavidin magnetic bead, without intervening capture of liposomes. By way of example, FIG. 2C illustrates a Poisson distribution relationship between digital signal (measured in percentage of fluorescing microwells out of all microwells of a plate) of imaged fluorescences against nucleotide copies per bead expressed logarithmically. Values of such a Poisson distribution can be generated by performing the above-mentioned quantitative-discrete PCR techniques according to example embodiments of the present disclosure, except that washed magnetic beads are incubated with biotinylated DNA rather than biotinylated liposomes.

The intermediate capture medium, for the purpose of this illustration, is made to bind specifically to DNA. Calibration bead populations were created by conjugating biotinylated DNA directly to streptavidin-coated beads (as illustrated in FIG. 2A) at 0.01, 0.1, 1, 10, or 100 copies per bead. For illustrative purposes, nucleic acid-bound beads enable assessment of method performance over a wide dynamic range and also serve as calibration standards for subsequent quantitation of vesicular payloads. Nucleic acid-bound bead populations are loaded into separate multiwelled plates (as illustrated in FIG. 2B) and analyzed by the above-mentioned quantitative-discrete PCR technique. The use of heat-labile biotin-streptavidin conjugation enables the DNA to release from the bead during a PCR hot-start step, so nucleic acids can be readily amplified in solution.

Reactor environments containing at least one copy of DNA produced high fluorescence signal after amplification, whereas reactor environments without DNA did not. PCR performance is characterized by measuring the digital signal (i.e., the percentage of “active” reactor environments, as described above) from each multiwelled plate. A concentration-dependent response is observed across the calibration range where digital signal increased with increasing DNA conjugation densities (as illustrated over the sloped portion of FIG. 2C). This illustrative, empirically measured digital response substantially accords with a theoretical signal predicted by a standard Poisson distribution, verifying that digital signal correlates well with theory across the calibration range. Furthermore, these results demonstrate that techniques as described herein allow deriving discrete per-vesicular manufacture loading measurements at low DNA copy numbers, without conflation with loading measurements taken from more than one liposome.

Analogous to the Poisson distribution as illustrated in FIG. 2C, a Poisson distribution relationship also exists between digital signal of imaged fluorescences against vesicular manufacture count as illustrated in FIG. 1C.

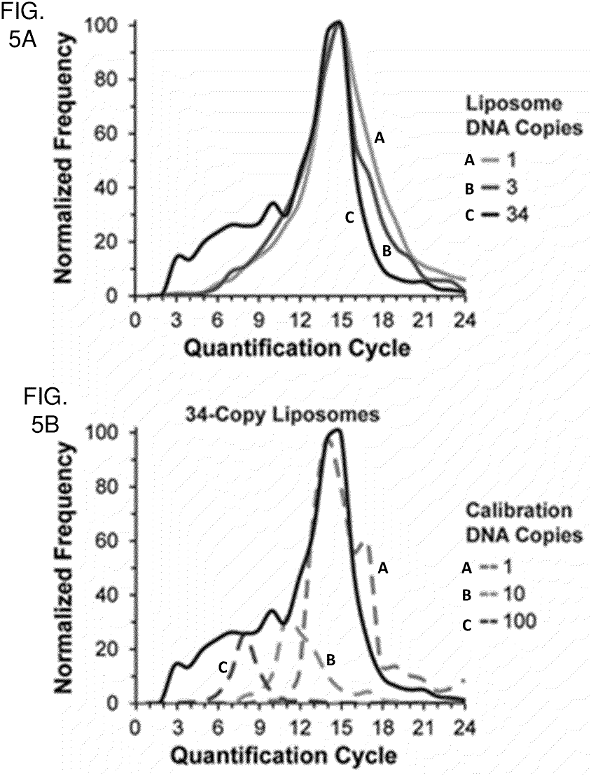

This correspondence in Poisson distribution can be demonstrated by measuring digital signals of imaged fluorescences for liposomes over a calibration range as described above. Concentration-dependent increases in digital signal are observed, demonstrating that bead capture of liposomes follows the Poisson distribution (as illustrated in FIG. 4A) in a similar manner as with free nucleic acid molecules.

Measuring digital signals over a calibration range furthermore demonstrates the quantitative capture of liposomes, and indicates a liposome concentration needed for single-vesicular manufacture analysis. Samples incubated at 80 M liposomes/mL produced a digital signal of 8.6%, which provides a >95% probability of active wells only containing a single liposome. Therefore, it can be understood that subsequent references to a “formed liposome population” can refer to populations with such liposome concentrations.

Moreover, when the intermediate capture medium captures liposomes rather than nucleic acid, PCR thermal cycles should produce sufficiently heated conditions to lyse the captured liposomes, prior to the first step of PCR amplification. Liposomes are expected to lyse at high temperature (i.e. 91° C.) during the PCR hot-start step; a time prerequisite to effectively lyse the vesicles is established as described below.

Bead-captured liposomes were analyzed according to PCR thermal cycles as described above, starting with a two-minute hot-start step. By qualitative observations of DNA being successfully amplified within the microwells, DNA can be observed as freed from vesicles during this hot-start time. A quantitative measurement of a digital signal as described above can further demonstrate digital signal being under the single-vesicular manufacture threshold as desired.

Upon further systematically decreasing hot-start times, no significant deviation in digital signal occurred with times down to 30 s, indicating effective lysis of the captured liposomes in this short time. Threshold C_{q} arrivals from active microwells (which can number in the several thousand on a single multiwelled plate) were aggregated into histograms and compared, resulting in no significant differences observed in either the average threshold C_{q} arrival or the FWHM.

Collectively, these results indicate that heat from PCR thermal cycle hot-starts effectively lyses liposomes within 30 s without the need for chemical denaturants, yielding uniform digital signal and uniform fluorescence intensity over time. This resulted in a complete set of PCR thermal cycles being completed in 11 minutes, which is significantly faster than PCR thermal cycles on comparable conventional equipment.

For further illustrative purposes, to verify that nucleotides detected by fluorescence originated from within liposomes and not outside, intact liposomes were incubated with and without DNase during the bead capture step to degrade unencapsulated DNA. No significant difference was observed between samples which indicates that the dialysis purification used after synthesis effectively removed unencapsulated DNA.

FIG. 1D illustrates a logarithmic signal curve graphing a sigmoidal relationship between the fluorescence intensity from each individual microwell and a number of PCR thermal cycles. Whereas FIG. 1C covers an entire multiwelled plate, FIG. 1D covers only each microwell of the multiwelled plate individually. The horizontal axis of FIG. 1D is effectively a time axis, only counted in number of cycles instead of time units.

According to the sigmoidal signal curve of FIG. 1D, a nucleic acid copy number is determined from the C_{q} overlaid upon the signal curve (illustrated as a horizontal broken line). Each cycle of a PCR reaction amplifies DNA from one sealed reactor environment where fluorescence increases after every PCR thermal cycle. A “threshold C_{q}” denotes a first cycle at which the analog fluorescence is distinguishable from the background signal, and a threshold C_{q} is constant for every individual fluorescence intensity signal curve over time. Samples with higher DNA copy numbers fluoresce after fewer PCR thermal cycles, and thus reach the threshold C_{q} earlier (represented by a sigmoidal signal curve further left on the horizontal axis), than those with fewer copies, which reach the threshold C_{q} later (represented by a sigmoidal signal curve further right on the horizontal axis).

As illustrated in FIG. 1D, three individual analog fluorescence intensity signal curves over time correspond to three categories of liposomes illustrated in FIG. 1A: liposomes labeled α contain a highest copy number of nucleotides, liposomes labeled β contain a smaller copy number, and liposomes labeled γ contain a smallest copy number. This is reflected in the relative positions of their respective sigmoidal analog fluorescence intensity signal curves.

The effects of these varying copy numbers are also seen in FIG. 1B. Over the course of an increasing number of PCR thermal cycles, reactor environments that captured a liposome labeled α fluoresce first; reactor environments that captured a liposome labeled β fluoresce next; and reactor environments that captured a liposome labeled γ fluoresce last out of the three categories.

For each distinct fluorescence profile among analog fluorescence intensity signal curves over time, multiple reactor environments can exhibit substantially the same distinct fluorescence profile. Therefore, by way of example, as illustrated in FIG. 1D, any count of reactor environments can exhibit the fluorescence profile α; any count of reactor environments can exhibit the fluorescence profile β; and any count of reactor environments can exhibit the fluorescence profile γ. Each count is proportional to a number of vesicle manufactures that were loaded with a particular copy number count, thereby indicating per-vesicle manufacture loading efficiency across the overall population. These fluorescence profile counts are further aggregated in FIG. 1E.

FIG. 1E illustrates a fluorescence profile count aggregation according to example embodiments of the present disclosure. Across the horizontal axis, fluorescence profiles can be organized from earliest to latest threshold C_{q} arrival, establishing some number of histogram bins, the number of bins corresponding to extent of heterogeneity in per-vesicle manufacture loading efficiency. The vertical axis of the histogram represents an aggregated fluorescence profile count for each distinct fluorescence profile.

C_{q} histograms were normalized to the most abundant value and scaled to 100%. Gaussian curves were fitted to the histograms using Igor Pro (Portland, OR) to characterize the centers and widths of the measured distributions. All analyses reported herein were performed in triplicate with n>25,000 total beads per sample. Error bars on plots represent ±1 standard deviation.

For illustrative purposes, such fluorescence profile count aggregations can be generated by performing the above-mentioned quantitative-discrete PCR techniques following from the above illustrative example wherein multiple populations of washed magnetic beads are incubated with biotinylated DNA in different respective copy numbers (rather than biotinylated liposomes), yielding multiple calibration bead populations.

For each calibration bead population, analog fluorescence was measured after every PCR thermal cycle performed upon a multiwelled plate. For each calibration bead population, distinct threshold C_{q} arrivals from each fluorescing reactor environment were sorted into aggregated fluorescence profile counts per reactor environment to visualize the population distribution (FIG. 2D, 0.01-copy population omitted for clarity). Curves were fit to each histogram to determine the average threshold C_{q} arrival and the threshold C_{q} arrival distribution of each calibration bead population.

As described above, given a single-vesicular manufacture threshold at 0.1 DNA copies per bead derived from the Poisson distribution, fluorescing reactor environments have a 95% probability of only containing a single copy of DNA. Thus, the threshold C_{q} arrival from this 0.1-copy data set (i.e., 16.8 PCR thermal cycles) denotes an average single-copy C_{q}.

According to established PCR techniques, a 3.3 cycle shift to earlier threshold C_{q} arrivals is expected for every 10-fold increase in DNA concentration. In practice, the average threshold C_{q} arrival from each bead population decreased by ~3 cycles between 1, 10, and 100 copies per bead, in accordance with performance expected of established PCR techniques.

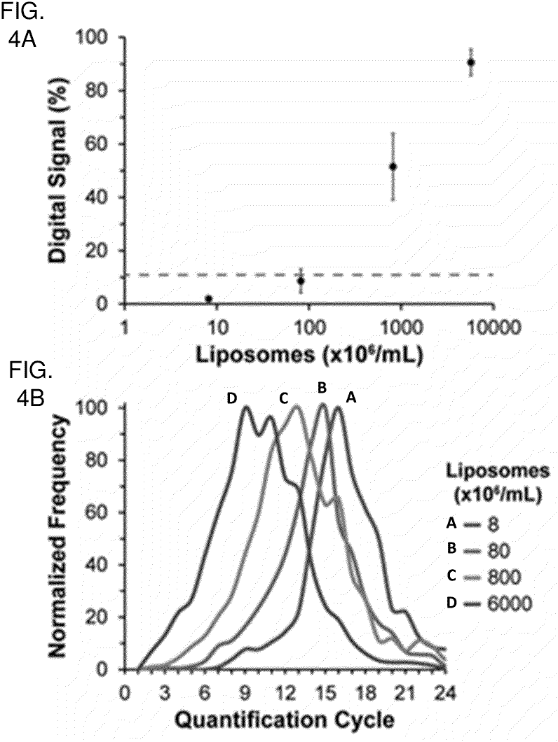

However, due to the stochastic nature of DNA conjugation to beads and inherent PCR efficiency differences between individual microwells, a substantially heterogeneous range of fluorescence profiles are observed among different reactor environments of a same multiwelled plate. FIG. 3A illustrates a series of fluorescence images showing one region of a multiwelled plate at increasing PCR cycle numbers. Bead autofluorescence is observed as white circles, while “active” reactor environments, exhibiting observable DNA fluorescence increases across different PCR thermal cycles. FIG. 3B illustrates a fluorescence profile count aggregation histogram.

Distributions are found to be broader at lower copy numbers because reactor-to-reactor differences in nucleic acid population are accentuated in single-vesicular manufacture analysis. However, the distribution of threshold C_{q} arrivals provides a definition of a threshold C_{q} arrival range for given copy numbers of unencapsulated nucleotides. The width of this distribution establishes a calibration baseline for measurements of per-vesicular manufacture nucleotide loading by quantitative-discrete PCR techniques as described above.

Threshold C_{q} arrival values are concurrently measured from each point across a calibration range as described above, causing aggregated fluorescence profile counts to shift to earlier threshold C_{q} arrival values at increasing liposome concentrations (as illustrated in FIG. 4B). This result is in line with predictions because multiple liposomes are captured onto individual beads at higher concentrations thus introducing more DNA copies into each microwell.

By way of example, at a high end of the calibration range (i.e. 6000 M liposomes/mL), the experimentally determined average liposomes/bead is 2.4 (i.e. 91% active). A Poisson distribution at this occupancy indicates between 1 and 6 liposomes are captured per bead, with each liposome adding ~10 additional DNA copies per microwell, and shifting the threshold C_{q} arrival earlier. Conversely, at lower values on the calibration range (i.e. 80 M liposomes/mL), the measured digital signal (i.e. 8.6% active) indicated single-vesicular manufacture capture.

Given that individual liposomes theoretically contain 10 DNA copies, a curve was fit to the aggregated fluorescence profile counts histogram and compared to model beads conjugated with 10 DNA copies. Average threshold C_{q} arrival values of liposomes and model beads were 16.3 and 11.7, respectively. This significant deviation shows that liposomes did not contain as many copies of DNA as intended. However, this is consistent with liposomes having smaller than intended diameters. Furthermore, assuming DNA encapsulation efficiencies ranging between 3 and 45% so loading fewer than 10 copies per liposome is expected.

Comparing the 16.3 threshold C_{q} arrival from liposomes to the 16.8 threshold C_{q} arrival from single-molecule model beads (i.e., copy number 0.1) indicates that individual liposomes contained 1-2 DNA copies. Considering the range of liposome volumes, this copy number corresponds to loading efficiencies of 10-20% for 100 nm liposomes or 33-67% for 70 nm liposomes.

The width of the aggregated fluorescence profile counts distribution can also be evaluated within or under a single-vesicular manufacture threshold, which showed a FWHM of 7.9 cycles as illustrated by the blue line of FIG. 4B. For comparison, the FWHM from the single-molecule model bead populations is 6.8 as illustrated by the blue line of FIG. 2B. The additional broadening of the liposome distribution compared to model beads is attributed to heterogeneity from the encapsulation of DNA within lipid nanoparticles.

Additionally, residual liposome components in a reactor environment may alter PCR efficiency and broaden the distribution. This contribution, however, is expected to be minor given the 10^5-fold larger volume of a microwell relative to a liposome.

Furthermore, as illustrated in FIG. 4B compared to FIG. 2D, liposomes exhibited a shoulder in the aggregated fluorescence profile counts histogram at earlier arrivals that is not present in the single-copy model beads. This shoulder correlates with model beads conjugated with 10 DNA copies. These results indicate heterogeneous packing where most liposomes contain 1 DNA copy but other liposomes contain up to 10 copies.

To ensure that liposome heterogeneity is not an artifact of the synthesis procedure, similar concentration-dependent responses are observed in both digital signals and analog fluorescence intensity signals over time for EA-formed liposomes as with FT-formed liposomes. These data demonstrate that DNA loading into liposomes is not significantly influenced by the synthesis method and that packing heterogeneity is prevalent in both populations.

Next, according to example embodiments of the present disclosure, digital signals and analog fluorescence intensity signals over time can be quantified from liposomes loaded with variable DNA copy numbers. Liposomes were manufactured using the FT procedure with expected DNA loading densities of 1, 3, or 34 copies in 70 nm liposomes. Liposomes from each population were then captured onto beads and analyzed on multiwelled plates.

Digital signals were imaged to validate operation within a single-vesicular manufacture threshold. The average digital signal was ~10%, which provided a ~95% probability that any active reaction environment captured only a single liposome. Within a single-vesicular manufacture threshold, subsequent threshold C_{q} arrival measurements will not suffer potential bias due to multiple liposomes being present in the same reaction environment.

Fluorescence intensity over time was measured to aggregate distribution of threshold C_{q} arrival values from DNA encapsulated within each liposome population, as illustrated in FIG. 5A. Results showed that the 1-copy and 3-copy liposome populations exhibited similar aggregated fluorescence profile counts histograms because of their similar DNA loading, with only a minor shift to earlier threshold C_{q} arrival values for the 3-copy liposomes. These distributions are similar to the 1-copy model beads as illustrated by the green line of FIG. 2D, which is consistent with expectations. However, a fraction of liposomes from both populations were loaded with up to 100 copies of DNA based on their early threshold C_{q} arrival values that overlapped with the 10-copy and 100-copy calibration beads.

Calibration beads are used to define a calibration threshold C_{q} arrival signal for each calibrator copy number; aggregated threshold C_{q} arrival signals for loaded vesicle manufacture populations can be compared against aggregated threshold C_{q} arrival signals for calibration beads, allowing estimation of the copy number loaded within.

The 34-copy liposomes did not exhibit the expected threshold C_{q} arrival shift of 3.3 cycles earlier than the other two populations. The center of the aggregated fluorescence profile counts distribution from 34-copy liposomes was aligned with the other populations; however, the tails of population differed. Rather than extend to later threshold C_{q} arrival values, the 34-copy liposomes tailed off sharply, which indicates that these liposomes are packed with more DNA than the other two populations, as expected.

Additionally, a shoulder was observed in 34-copy liposomes at early threshold C_{q} arrival values signifying high DNA loading. Threshold C_{q} arrival values in this range are higher than model beads conjugated with 100 copies, as illustrated by FIG. 5B.

As illustrated in FIG. 5B, the distribution of threshold C_{q} arrival values in liposomes (black) are compared to those from the calibration beads (red, orange, green). We know liposomes in this population contain between 1 and 1000 DNA copies based on comparing their threshold C_{q} arrival values to the threshold C_{q} arrivals from the calibration beads.

Techniques herein quantify extent of loading efficiency heterogeneity per-vesicle manufacture in a vesicle manufacture population, which cannot be accomplished with bulk analyses. Such heterogeneous loading as observed among the 34-copy liposomes can be attributed to multiple compounding sources of variability arising from the FT synthesis and extrusion steps.

These results suggest that a subset of liposomes was packed with hundreds to thousands of DNA copies. Particle sizing data did not show an increase in liposome aggregation in this population, meaning that this dense DNA loading arose from the synthesis. The identification of some liposomes becoming excessively enriched with DNA provides information which quality control technicians can act upon to inform synthesis procedures by identifying freeze-thaw conditions that promote more uniform liposome packing.

Individual liposomes in the synthesis mixture undergo freezing and thawing at different times and become embedded at different depths within ice crystals, thus having different access to encapsulate the DNA in solution. Furthermore, cryo-concentration can occur during the freezing process where molecules enrich at the interface between ice crystals, creating spatially heterogenous DNA distributions.

Based on such observations, during thawing, liposomes adjacent to cryo-interfaces encapsulated the high-concentration DNA solution could become highly loaded, whereas liposomes entrapped in bulk ice could only load few DNA copies. Reconciling the mechanisms of FT liposome loading with this data explains could explain observations of a relatively small fraction of liposomes highly loaded with DNA while most liposomes have low DNA loading.

Heterogenous particle loading — where a fraction of liposomes are highly enriched — in biopharmaceutical formulations can elicit different biological activity including potentially detrimental side effects. The improved post-process quality control measurements of per-vesicular manufacture loading efficiency and encapsulation efficiency in vesicular biologics, by quantitative-discrete PCR techniques as provided herein, can guide synthesis processes, whether synthetic or biogenesis in nature, to yield more homogenous formulations.

Furthermore, a quantitative-discrete PCR technique according to example embodiments of the present disclosure eliminates shortcomings of conventional PCR techniques which can introduce biases, false positives, and false negatives when quantifying nucleic acid-based biologics, including any varieties of coding RNA molecules, as well as non-coding nucleic acids as described above. For example, according to ligase-dependent, reverse-transcription quantitative PCR (“RT-qPCR”) methods, miRNAs are first ligated with poly adenosine using a Poly A polymerase enzyme to extend the miRNA sequences. The extended miRNAs are then reverse transcribed into complimentary DNAs (“cDNAs”) using a reverse transcriptase enzyme. cDNAs can then be amplified by PCR using a DNA polymerase enzyme. Fluorescent intercalating dyes are used to detect the double-stranded DNA products, whose signals are measured after each thermocycle. As described above, a first cycle at which the fluorescence intensity signal is detectable above the background is designated as the threshold C_{q}. The higher the concentration of cDNA after reverse transcription (RT), the earlier the threshold C_{q} because more DNA molecules are available to produce a fluorescence signal for detection. C_{q} values are calibrated to measure cDNA concentrations, which correspond to the miRNA concentrations in the original sample.

However, such multiple sample preparation steps and distinct enzymes required for RT-qPCR result in increased cost and complexity. Additionally, systematic ligase-dependent biases can occur: sequence-specific miRNA conformations can influence ligation, resulting in under- or over-representation of certain miRNAs in the total cDNA products. Moreover, the qPCR process can self-bias in the event that a single contaminant or mis-priming event is amplified, leading to false positives. Similarly, the presence of a PCR inhibitor can hinder the PCR reaction, leading to false negatives. Such sources of error can all adversely impact qPCR measurements.

Stem loop RT-qPCR techniques bypass miRNA ligation by extending miRNA sequences using stem loop primers are used to extend miRNA sequences. Still, such techniques do not circumvent the RT sample preparation step.

Digital PCR (“dPCR”) is an alternative technique to quantify miRNAs. dPCR first partitions a sample into >10³ discrete ultralow-volume reactions. PCR is then performed on all partitions in parallel. Partitions containing even a single target molecule are amplified in the ultralow volume to produce a readily detectable florescence signal; these partitions are classified as digitally “active”. Partitions without a target molecule remain nonfluorescent after PCR and are classified as digitally “inactive”. The percentage of active partitions is proportional to the analyte concentration in the original sample. Although dPCR provides additional analytical benefits, it still requires the multiple sample preparation steps and enzymes as qPCR to produce a detectable signal.

A quantitative-discrete PCR technique according to example embodiments of the present disclosure utilizes base-stacking, obviates ligation and reverse transcription steps required by conventional PCR techniques, utilizes amplification reactions within discrete reactors, optimizes thermocycling conditions to improve amplification specificity and minimize non-specific amplification, and minimizes sample preparation processes.

Nuclease-free water, DNA guide, let-7a miRNA, and PCR primers, as listed in Table 1 below, were purchased from Integrated DNA Technologies (Coralville, IA).

TABLE 1

| | |
|---|---|
| Reagent | Sequence (5′-3′) |
| DNA Guide | Biotin-GGCTAAGACAGATGCTCTTTGCCAACAGGCCACAGAA TTCCTACACTAAAAGTCGTACTGAACTATACAACCTACTACC TCATCGCACT (SEQ ID NO: 1) |
| let-7a miRNA | UGAGGUAGUAGGUUGUAUAGUU (SEQ ID NO: 2) |
| Forward Primer | TACGAGAGATGCGA (SEQ ID NO: 3) |
| Reverse Primer | GGCTAAGACAGATGCTC (SEQ ID NO: 4) |

A quantitative-discrete PCR technique is performed to quantify a target nucleic acid. By way of example, let-7a miRNA was selected here as a target nucleic acid because of its roles in cell proliferation and various disease pathways.

PCR master mix, as described in Table 2 below, was obtained from KAPA Biosystems (Wilmington, MA).

TABLE 2

| | | |
|---|---|---|
| Reagent | Components | Concentration |
| Microfluidic PCR master mix | KAPA SYBR Fast mix | 1x |
| | SYBR Green | 5x |
| | MgCl₂ | 2 mM |
| | Primers | 6 µM |
| | Kapa 2G Polymerase | 0.15 U/µL |
| Bulk commercial PCR master mix | KAPA SYBR Fast mix | 1x |
| | Rox Reference Dye | 100 nM |
| | DNA Guide | 200 nM |
| | Primers | 200 nM |

Tris-hydrochloride, 10x tris-buffered saline (TBS), 10% bovine serum albumin (BSA), Tween 20, Dynabeads M-270 streptavidin, ethanol, 10,000x SYBR Green, trichloro(octyl) silane, and 5% buffered oxide etchant were purchased from ThermoFisher Scientific (Waltham, MA). Hexamethyldisilazane (HMDS) was obtained from Integrated Micro Materials (Argyle, TX). PRS 2000 was purchased from Mays Chemical (Indianapolis, IN). Ammonium hydroxide and hydrogen peroxide were obtained from Millipore Sigma (St. Louis, MO). Vacuum pump oil was acquired from VWR (Radnor, PA).

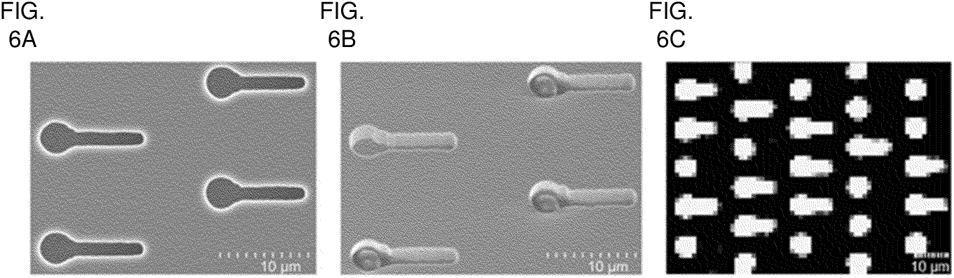

Microwell features were patterned onto silicon wafers (University Wafer, Boston, MA) using photolithography. Wafers were then subjected to deep reactive ion etching (SPTS Technologies, Newport, UK) to create recessed wells in the silicon. The final depths of the microwells were measured to be 4 µm using a profilometer (Bruker, Billerica, MA). Scanning electron microscope (SEM) (JEOL, Peabody, MA) images, as illustrated in FIG. 6A, showed that the microwells had 3 µm bead-loading circles, as illustrated in FIG. 6B, adjoined to 8 × 1.3 µm signal acquisition rectangles.

Fluidic domes were patterned onto glass (S.I. Howard Glass, Worcester, MA) by photolithography and then etched to a depth of 20 µm using buffered oxide etchant. Glass and silicon substrates were cleaned in a base piranha solution (5 : 1 : 1 water : ammonium hydroxide : 30% hydrogen peroxide) for 15 min at 60° C. Substrates were then baked for 10 cmin at 110° C. and cleaned with an oxygen plasma (Plasma Etch, Carson City, NV). Both substrates were silanized with trichloro(octyl) silane under vacuum at 110° C. for 1 h. Glass and silicon substrates were then diced into individual units and epoxied together to form the final microwell array devices, as illustrated in FIG. 6C.

In accordance with techniques described above with reference to FIGS. 2A through 2D, biotinylated DNA guides were conjugated onto streptavidin-coated beads (2.8 µm diameter). The conjugation density of DNA guides was varied between 3×10³ and 3×10⁶ molecules per bead. Microfluidic microwell arrays were conditioned with ethanol, water, and loading buffer, as described in Table 3 below.

TABLE 3

| Reagent | Components | Concentration |
|---|---|---|
| Loading buffer | Tris HCl, pH 8 | 20 mM |
| | KCl | 20 mM |
| | MgCl₂ | 2.5 mM |
| | Tween 20 | 0.10% |
| | BSA in 1x TBS | 1% |

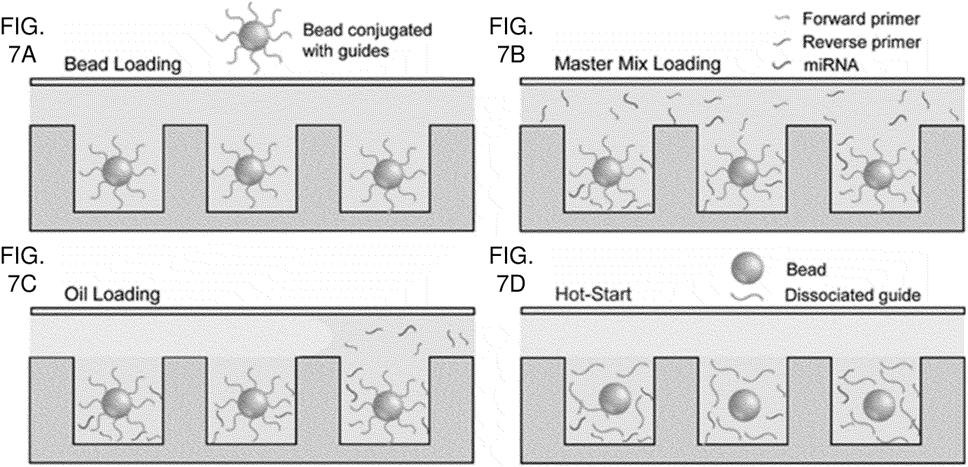

Guide-conjugated beads were then loaded into array devices using a magnet to draw beads into the microwells, as illustrated in FIG. 7A. Microwells were engineered with dimensions to only contain a single bead. The volumes of a microwell and a bead are 80 fL and 10 fL, respectively. PCR master mix was vacuumed into devices and allowed to sit for 3 min to enable master mix to passively diffuse into the microwells, as illustrated in FIG. 7B. Loaded microwells were then sealed with oil to create discrete reaction partitions, as illustrated in FIGS. 7C and 7D.

According to quantitative-discrete PCR as described herein, PCR was performed on an AZ100 epifluorescence microscope (Nikon Instruments, Melville, NY) where the microscope stage is mechanically converted to electrically function as a thermal cycler apparatus. The PCR thermocycling program contained a 30-s hot-start step at 94° C. (FIG. 7D), a 6-s melting step at 94° C., a variable annealing step (reported in the Results and Discussion), and a 10-s extension step at 69° C. A total of 35 PCR cycles were performed for each analysis. Fluorescence images of devices (FIG. 2C) were acquired after every PCR thermocycling step for 1 s at 470/525 nm and 8x magnification. An Andor Zyla sCMOS camera (Oxford Instruments, Smyrna, TN) and a Sola SE light source (Lumencor, Beaverton, OR) were used to collect the images. Digital signal was calculated as the percentage of active microwells at the end of the 35 cycles. Microwells were designated as active if their fluorescence was observable above the device background (S/N>1.03). PCR signal curves were obtained by plotting the average signal from all active microwells at each PCR thermocycle. All analyses were performed in triplicate with n>10,000 total analyzed beads per replicate. In each Figure plotting analysis results (as shall be subsequently described), the average signal from these n=3 analyses with error bars depicting ±1 standard deviation.

As a comparative reference, conventional PCR was performed on a QuantStudio™ 12 K Flex Real-Time PCR System (ThermoFisher Scientific). 20 µL of PCR master mix (as described in Table 2 above) was added into a MicroAmp Optical 96-Well Reaction Plate (ThermoFisher Scientific). A thermocycling program was performed including a 180-s hot-start step at 95° C., 40 cycles of a 1-s melting step at 95° C., and a 30-s combined annealing and extension step at 60° C. Signal curves at each let-7a concentration were measured in triplicate, and normalized to the lowest fluorescent intensity. C_{q} values were automatically determined by the instrument software.

Furthermore, according to quantitative-discrete PCR as described herein, PCR reactions are performed by the assembly, in each reactor environment, of a three-component base-stacking complex: a single-stranded DNA guide with a complementarity region to the target nucleic acid, the target nucleic acid, and a forward primer with only five complimentary nucleotides to the DNA guide. In the absence of the target nucleic acid, the forward primer does not have sufficient binding energy to anneal to the DNA guide. However, hybridization of the target nucleic acid onto the DNA guide stabilizes annealing of the forward primer to the DNA guide. This occurs due to pi-pi stacking interactions between the 3′ end of the primer and the 5′ end of the target nucleic acid. This base-stacking complex is then extended by a DNA polymerase to form a full-length amplicon, which can be amplified in subsequent PCR cycles.

According to the present example, a DNA guide was designed with a region fully complementary to let-7a to enable hybridization. The guide was also complementary to the terminal five nucleotides on the 3′ end of the forward primer. The guide and primer sequences were modified from those reported in the literature after screening with Visual Oligonucleotide Modeling Platform (DNA Software, Plymouth, MI) to improve binding efficiency.

Various parameters of a quantitative-discrete PCR as described herein can be further optimized. PCR reactions may perform unexpectedly due to the small reactor environment volume (as embodied by microwell and bead volumes as mentioned above), wherein higher reagent concentrations to ensure sufficient numbers of molecules are loaded into each discrete reaction partition. Furthermore, PCR reactions may perform unexpectedly due to high surface area-to-volume ratio in discrete reactor environments, which tends to accentuate surface adsorption issues that may cause PCR inhibition.

By way of example, a number of DNA guides available in each reaction can be optimized, to ensure robust formation of the base-stacking complex. More DNA guides per reaction increases the efficiency of the PCR reaction by providing more opportunities for the target nucleic acid and forward primer to hybridize to a DNA guide and trigger RT-free amplification. However, too many DNA guides leads to non-specific amplification, causing false positive signals. An optimized number of DNA guides can maximize on-target signal while minimizing non-specific amplification.

Rather than adding guides directly into the PCR master mix, guides were biotinylated and conjugated to streptavidin beads to enable their controlled delivery into the microwells. A two-tiered signal response was sought for method optimization, where all microwells were either active or inactive. To achieve this, positive controls were prepared by adding let-7a to the PCR master mix at 4 copies of target nucleic acid per microwell. Negative controls only contained master mix without target nucleic acid. Positive and negative controls were expected to provide 98% and 0% digital signal, respectively, as predicted by Poisson distribution.

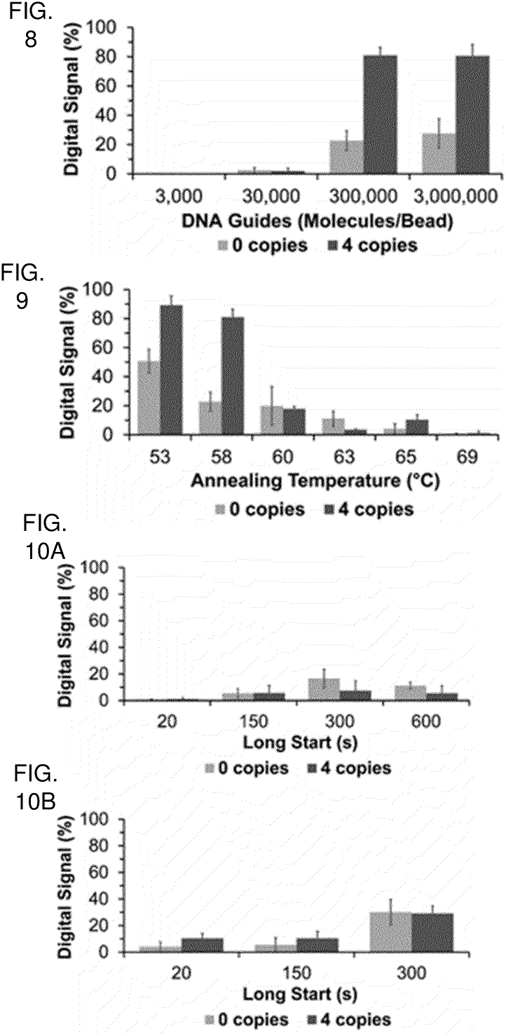

Digital signal was measured from positive and negative controls using between 3×10³ and 3×10⁶ guides/bead. At 3,000 and 30,000 guides/bead, the percentage of active wells for both positive and negative controls were near zero and indistinguishable from each other, as illustrated in FIG. 8. Digital signal increased when using 300,000 guides/bead but then plateaued, with no further improvements at 3,000,000 guides/bead. The highest ratio between the digital signal from the positive control and negative control (“P/N ratio”) occurred using 300,000 guides/bead.

Furthermore, by way of another example, annealing temperature is optimized. False positives can result from non-specific amplification, yielding up to 100% activity signals and therefore substantially obfuscating result readings. Non-specific amplification signals can result even according to optimized copy numbers as derived above. Due to the relatively high reagent concentrations in discrete reactor environments, transient hybridization of the primer to the guide occurs at a sufficient frequency to be amplified; non-specific priming of the DNA guides may be occurring even in the absence of target nucleic acid. This result is surprising given that the forward primer only has five complementary nucleotides to the DNA guide, and given that melting temperature of primer-guide binding is computationally predicted to be -5° C. (VisualOMP).

During the annealing step, both the target nucleic acid and forward primer must hybridize to the DNA guide. Given the high rates of false positives observed as discussed above, non-specific binding between the forward primers and DNA guides in the absence of target nucleic acid likely occurred because the annealing temperature was too low. However, annealing temperatures should be finely tuned as excessively high temperatures can decrease PCR yield by preventing on-target hybridization and negatively affect the efficiency of the assay.

Thermocycling programs with annealing temperatures between 53° C. and 69° C. were evaluated using 300,000 guides/bead. Digital signals for both positive and negative controls were observed to decrease as annealing temperature increased. Binding between primers and guides became less stable at higher temperatures, lowering the probability of a successful extension event, as illustrated in FIG. 9. While digital signal from the negative control decreased gradually over this temperature range, signal from the positive control decreased sharply at 60° C., suggesting dissociation of the on-target base-stacking complex. The highest P/N ratio occurred at 58° C.; however, the positive control still underperformed its predicted 98% activity because of inefficient hybridization between the DNA guide, target nucleic acid, and forward primer. Similarly, the negative control exhibited prohibitively high digital signal at 58° C., which was attributed to the extension of transient primer-guide hybrids due to the high reagent concentrations in the microwells.

A high rate of false positives was observed at all temperatures, except 69° C. where signal from the positive control was near zero and indistinguishable from the negative control.

By way of another example, according to example embodiments of the present disclosure, asymmetric thermocycling is performed to increase the signal from the positive control without increasing signal from the negative control. Rather than keeping the time and temperature of the melting, annealing, and extension steps constant throughout each cycle, parameters of the first cycle are different from parameters of subsequent cycles, tailored to promote base-stacking complex formation, with the goal of increasing P/N ratio and promoting on-target amplification. The complex can be assembled in Cycle 1 to be extended by the polymerase and produce a full-length amplicon. Once this product has been produced, it can be amplified under non-tailored PCR reaction parameters in Cycles 2-35, to reduce the probability of non-specific amplification in subsequent cycles.

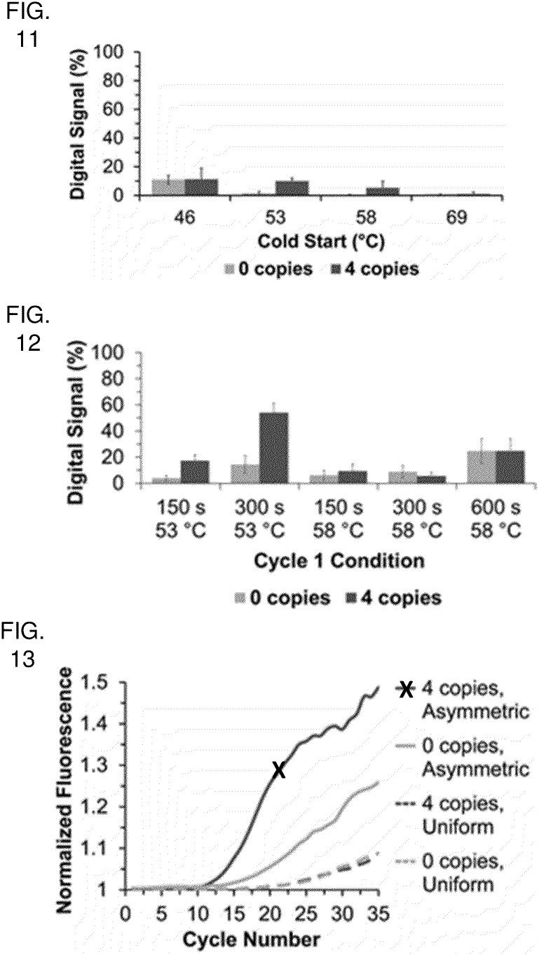

The Cycle 1 annealing time was first evaluated to determine if a long-start could increase on-target PCR amplification. The results showed that the digital signal was indistinguishable between positive and negative controls with a long-start up to 600 s, as illustrated in FIG. 10A. A 69° C. annealing temperature was used for these experiments because the negative control in the study above did not produce signal, but this temperature may have been prohibitively high. Therefore, a second long-start study was performed at an annealing temperature of 65° C. to make hybridization more favorable. However, similar trends were observed where digital signals from positive and negative controls were indistinguishable, regardless of the annealing time at Cycle 1, as illustrated in FIG. 10B. These results demonstrated that simply providing more time for the base-stacking complex to form was insufficient to enhance PCR amplification efficiency.

Next, the effect of decreasing the Cycle 1 annealing temperature was investigated to promote formation of the base-stacking complex. Among cold-starts between 46° C. and 69° C., two conditions at 53° C. and 58° C. produced higher digital signal for positive controls than for negative controls, as illustrated in FIG. 11. However, on-target signal remained significantly lower than expected, suggesting that lowering the Cycle 1 annealing temperature alone was insufficient to increase the response of the positive control. However, when both cold-start and long-start were combined into a single program (subsequently a “cold-long-start” thermocycling program), an improved response was observed, as illustrated in FIG. 12. The program using a 300-s long-start combined with a 53° C. cold-start achieved the greatest performance, with a P/N ratio of 3.8. This suggests that the base-stacking complex requires both longer times and colder temperatures to promote assembly of the guide, target nucleic acid, and primer. However, even with this enhanced performance, positive controls remained underactive compared to their theoretical values while negative controls were overactive.

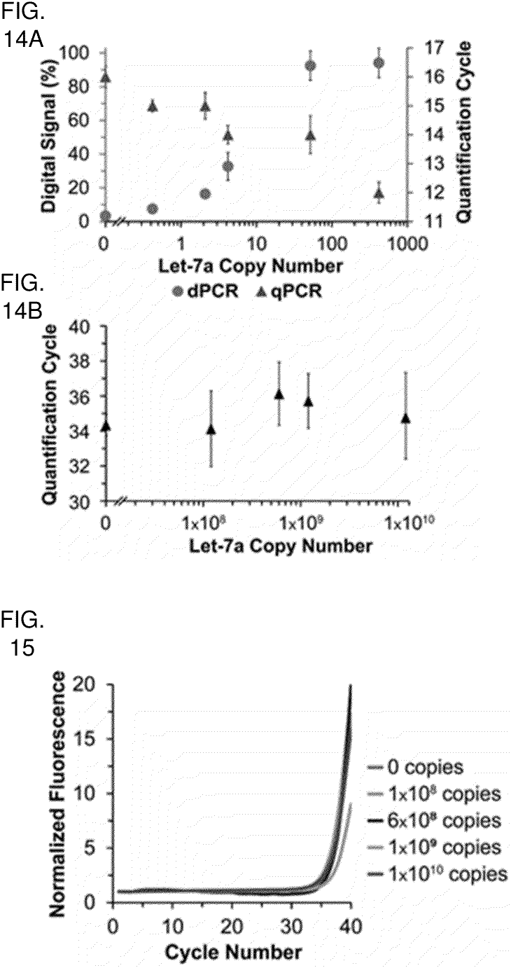

Signal curves of active microwells from positive and negative controls were obtained for the best uniform (annealing at 58° C. for 20 s) and asymmetric (Cycle 1 annealing at 53° C. for 300 s and Cycles 2-35 at 69° C. for 20 s) thermocycling programs. Under conventional uniform thermocycling, the negative and positive controls were indistinguishable from each other, having the same threshold C_{q} of 27, as illustrated in solid lines of FIG. 13. However, using the cold-long-start thermocycling program, the positive control exhibited a threshold C_{q} of 13, as illustrated in broken lines of FIG. 13, shifting 14 cycles earlier compared to uniform thermocycling. This C_{q} shift indicated more efficient amplification, demonstrating the benefits of the cold-long-start program. The negative control also experienced a C_{q} shift with a cold-long-start, but the majority of active microwells appeared at later cycles. These false positives could be eliminated from the assay by ending PCR at Cycle 17.

Although some active microwells from the positive control are sacrificed, conducting fewer PCR thermocycles increased the P/N from 3.8 to 9.7 and reduced the analysis time to 15 min. Assays using the cold-long-start program also exhibited active wells with higher analog fluorescence. This higher signal-to-noise enabled digitally active and inactive microwells to be more easily assigned and aided in the determination of C_{q} values.

The optimized quantitative-discrete PCR technique above produced a high P/N ratio, although digital signals were lower than theoretical predictions. Such results suggest incomplete diffusion of target nucleic acid from the bulk master mix solution into the microwells over the short time provided for device loading. However, even if reagent concentrations did not reach equilibrium between the bulk and the microwells, proportional concentration-dependent responses should still be observed.

Thus, the quantitative performance of quantitative-discrete PCR was evaluated by analyzing a series of target nucleic acid concentrations using the optimal asymmetric thermocycling program established above (Cycle 1 annealing at 53° C. for 300 s and Cycles 2-35 at 69° C. for 20 s). Digital signals were found to increase between 0 and 50 copies per microwell (FIG. 14A, circles), proving digital signal obtained from techniques as described herein is concentration dependent. No digital signal change was observed between 50 and 500 copies/microwell because the digital signal saturated at these high concentrations. However, 50 and 500 copies/microwell were discernable using C_{q} values, as illustrated by triangles in FIG. 14A. A two-cycle shift in C_{q} was observed between 50 and 500 copies of let-7a per microwell. This is below the expected three-cycle shift, but the general trend of higher target nucleic acid copy numbers shifting C_{q} values to earlier cycles matches with theoretical predictions.

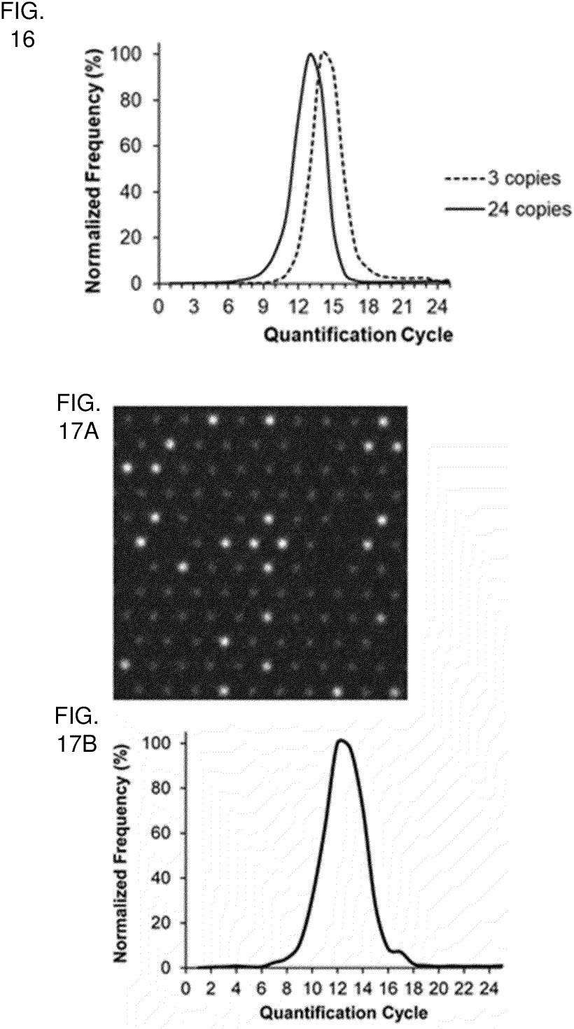

As a benchmark, bulk results were derived from a commercial qPCR instrument. C_{q} values were measured from samples containing between 0 and 10¹⁰ let-7a copies per reaction (as illustrated in FIG. 15) based on the concentrations used in the above-described processes. All C_{q} values from bulk qPCR assays were indistinguishable from one another and from the blank, as illustrated in FIG. 14B. This suggests that signal obtained from bulk qPCR was not produced by the let-7a, but rather from mis-priming events between DNA guides and forward primers. These results show that bulk qPCR did not yield a concentration-dependent response to the target nucleic acid. They also highlight the good results obtained from microfluidic quantitative-discrete PCR, as our on-chip technique demonstrated a more sensitive and quantitative method to measure target nucleic acid at low copy numbers.

The above-described techniques, as applied to coding RNA molecules as well as to non-coding nucleic acids, eliminated the need for ligation and RT steps, which reduced the time, complexity, and cost of the analysis compared to conventional qPCR methods. The success of microfluidic quantitative-discrete PCR stemmed from an asymmetric PCR thermocycling program which facilitates the formation of the base-stacking complex while concurrently minimizing non-specific amplification. Additionally, using analog fluorescence intensity signals to inform digital signal analysis resulted in the optimized method providing a high P/N ratio. Incorporating both digital signal and analog fluorescence intensity signals over time into a single assay afforded an ultrawide dynamic range, where the digital signals reliably measured low copy numbers, but at high copy numbers where digital signal saturated, C_{q} values derived from analog fluorescence intensity readily discerned sample concentrations. Overall, the short analysis times and high sensitivity demonstrate that microfluidic quantitative-discrete PCR can analyze coding RNA molecules and non-coding nucleic acids for post-process quality control of vesicular biologics. Thus, the above-described techniques can be performed in conjunction with the capture of vesicular manufactures onto an intermediate capture medium in order to perform single-vesicular manufacture analysis as described herein.

Furthermore, quantitative-discrete PCR techniques according to example embodiments of the present disclosure can be demonstrated to quantify the number of target nucleic acids loaded into individual vesicular manufactures by PCR in single-vesicular manufacture analysis, without the need for ligation or reverse transcription steps. By way of example, the number of miRNA molecules loaded into individual lipid nanoparticles (LNPs), where LNPs are an example of vesicular manufactures, can be measured by capture onto an intermedia capture medium as described below.

Two populations of LNPs were synthesized to theoretically contain either 3 or 24 copies of the miRNA miR-146a. LNPs were then captured onto beads which were first conjugated with 100,000 DNA guides per bead required for the PCR reaction. The beads conjugated with DNA guides is an example of an intermediate capture medium which is not bioreceptor-bound. Prepared beads were loaded into microwell array devices and sealed to form individual reaction chambers. PCR thermocycling was performed using a program containing a hot-start step at 95° C. for 30 s, a cold-long-start step at 48° C. for 5 min, and 19 PCR cycles with a 6 s 93° C. melt step, a 15 s 58° C. annealing step, and a 15 s 68° C. extension step. The heat from the PCR hot-start step was used to simultaneously activate the DNA polymerase and lyse LNPs. This liberated the internal miRNA cargo from LNPs into their own individual sealed microwells. Microwell environments were prepared to form base-stacking complexes therein including a DNA strand and a forward primer in the presence of the miRNA, obviating the need for ligase and reverse transcriptase enzymes which are typically required for miRNA PCR. This streamlined approach used heat as a chemical-free means of lysing LNPs without concerns about deactivating these non-heat-stable enzymes. The cold-long-start program then enabled miRNAs to be amplified using only a DNA polymerase.

The C_{q} was measured from individual microwells containing a LNP. C_{q} values were sorted into histograms to reveal the heterogeneity of miRNA loading within the LNP populations. The results showed miRNA was loaded uniformly in the populations, as illustrated in FIG. 16, without the significant heterogeneity observed with DNA as illustrated in FIG. 5B. LNPs packaged with 24 copies of miRNA exhibited a shift to earlier Cq values on average compared to LNPs packaged with 3 copies, which is consistent with higher RNA loading. This data demonstrates that the techniques described herein can measure non-coding RNAs within a population of individual LNPs, which expands its utility for pharmaceutical applications.

Moreover, example embodiments of the present disclosure are applicable to vesicular manufactures derived by biogenesis pathways. By way of example, vesicles were isolated from cell culture media using an exosome isolation kit (ThermoFisher). The vesicles were then transfected with the miRNA let-7a using an exosome transfection kit (System Biosciences). Samples were purified to remove unencapsulated miRNA.

Streptavidin beads were prepared with both biotinylated DNA guides and biotinylated capture antibodies as bioreceptors. The beads conjugated with DNA guides and biotinylated capture antibodies is an example of a bioreceptor-bound intermediate capture medium. DNA guides for let-7a were conjugated at 100,000 guides per bead. Those same beads were then saturated with capture antibody selective for the vesicle membrane protein CD63. The final bioreceptor-bound beads are conducive to capturing vesicles from biological samples or pharmaceutical formulations and conducting RT-free RNA PCR in sealed microwells.

Bioreceptor-bound beads were incubated with the solution of miRNA-transfected vesicles. Vesicles were selectively captured onto the bioreceptor-bound beads via CD63 antibodies. A low vesicle concentration was used to ensure capture in the single-vesicle regime. Prepared beads were then loaded into an array of cylindrical microwells as illustrated in FIG. 17A, and sealed with oil to form discrete reaction chambers for PCR analysis.

A cold-long-start PCR program as described above was used for analysis. The thermocycling program employed a hot-start step at 95° C. for 30 s, a cold-long-start step at 53° C. for 5 min, and 19 PCR cycles with a 6 s 93° C. melt step, a 15 s 58° C. annealing step, and a 15 s 68° C. extension step. The high temperature from the hot-start step first lysed the vesicles, which liberated the encapsulated miRNA molecules into the sealed reaction chambers. C_{q} values were measured from each active microwell to quantify miRNA loading within individual vesicles. FIG. 17B illustrates the fluorescence profile count aggregation of the resulting data. The curve indicates a relatively Gaussian distribution of miRNA loaded into a population of vesicles. However, a small shoulder was observed at later cycles, which indicates that a small percentage of vesicles were underloaded.

It should be understood that the example of FIGS. 17A and 17B is specific to a particular nucleic acid copy number per vesicle. Further calibration bead populations for other nucleic acid copy numbers per vesicle can be prepared as described above with reference to FIGS. 2C and 2D.

These results demonstrate rapid, RT-free, one-pot measurements of non-coding RNA molecules from individual vesicles. Thus, quantitative-discrete PCR techniques according to example embodiments of the present disclosure can be applied to vesicular manufactures yielded from synthetic and biogenesis pathways alike, and can be expanded to measuring endogenous nucleic acid packaging from cell-derived vesicles to support biomedical research studies.

Although the subject matter has been described in language specific to structural features and/or methodological acts, it is to be understood that the subject matter defined in the appended claims is not necessarily limited to the specific features or acts described. Rather, specific features and acts are disclosed as exemplary forms of implementing claims.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster’s Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method comprising:
capturing a formed vesicular manufacture population onto an intermediate capture medium, the formed vesicular manufacture comprising a target nucleic acid;
capturing the intermediate capture medium in a plurality of sealable reactor environments;
measuring a digital signal comprising a percentage of fluorescing reactor environments after at least one polymerase chain reaction (PCR) thermal cycle; and
measuring a fluorescence intensity from each reactor environment after each of a plurality of PCR thermal cycles.

2. The method of claim 1, wherein the intermediate capture medium comprises a bioreceptor-bound magnetic bead.

3. The method of claim 2, wherein the bioreceptor comprises a biotinylated capture antibody.

4. The method of claim 2, wherein the bioreceptor comprises a biotinylated lipid.

5. The method of claim 1, wherein each reactor environment is configured to capture no more than one intermediate capture medium.

6. The method of claim 1, wherein the at least one PCR thermal cycle comprises a hot-start step 30 seconds in duration.

7. The method of claim 6, wherein the vesicular manufacture population is substantially lysed during the hot-start step.

8. The method of claim 7, wherein each lysed vesicular manufacture expels respective loaded contents into a respectively sealed reactor environment.

9. The method of claim 1, wherein the digital signal is within a single-vesicular manufacture threshold.

10. The method of claim 1, wherein fluorescence intensities from a plurality of reactor environments exhibit a plurality of fluorescence profiles each occurring with a respective count.

11. The method of claim 10, further comprising aggregating a plurality of fluorescence profile counts among several thousand measured fluorescence intensities.

12. The method of claim 10, wherein the plurality of fluorescence profile counts are aggregated across fluorescence profile bins sorted from earliest to latest PCR thermal cycle of threshold quantification cycle (C_{q}) arrival, wherein a threshold C_{q} is a PCR thermal cycle at which fluorescence is distinguishable from background signal.

13. The method of claim 1, wherein the reactor environment comprises a single-stranded DNA guide complementary to the target nucleic acid, and a forward primer complementary to the DNA guide.

14. The method of claim 13, wherein the reactor environment comprises at least 100,000 DNA guides.

15. The method of claim 13, wherein a PCR thermal cycle is performed at an annealing temperature of 69° C.

16. The method of claim 13, wherein a first PCR thermal cycle comprises a hot-start step at a temperature effective to activate a PCR polymerase and to lyse a formed vesicular manufacture population, the first PCR thermal cycle is performed at an annealing temperature of 53° C. for 5 min, and each subsequent PCR thermal cycle is performed at an annealing temperature of 58° C. for 15 s.

17. The method of claim 13, wherein no more than seventeen PCR thermal cycles are performed.

18. A method comprising:
manufacturing a formed liposome population from biotinylated lipids;
capturing a formed liposome population onto streptavidinylated beads, the streptavidinylated beads being bound with biotinylated capture antibodies;
capturing the streptavidinylated beads in a plurality of microwells etched into a multiwelled plate, each microwell of the plurality of microwells comprising a capture subwell having a substantially spherical form, and a reactor subwell having a substantially elongated form narrower in width than a diameter of the capture subwell, wherein the capture subwell is configured to capture no more than one streptavidinylated beads and the reactor subwell is configured to deny entry of streptavidinylated beads;
measuring a digital signal comprising a percentage of fluorescing microwells among the plurality of microwells after at least one polymerase chain reaction (PCR) thermal cycle;
measuring a fluorescence intensity from each microwell of the fluorescing microwells after each of a plurality of PCR thermal cycles; and
aggregating a plurality of fluorescence profile counts in a histogram across fluorescence profile bins sorted from earliest to latest PCR thermal cycle of threshold quantification cycle (C_{q}) arrival, wherein a threshold C_{q} is a PCR thermal cycle at which fluorescence is distinguishable from background signal;
wherein the reactor environment comprises at least 100,000 single-stranded DNA guides complementary to the target nucleic acid, and a forward primer complementary to the DNA guide;
wherein a first PCR thermal cycle comprises a hot-start step at a temperature effective to activate a PCR polymerase and to lyse a formed liposome population, the first PCR thermal cycle is performed at 53° C. for 300 s, and each subsequent PCR thermal cycle is performed at 69° C. for 20 s; and
wherein no more than seventeen PCR thermal cycles are performed.


SEQUENCE LISTING (4 sequences)

SEQ ID NO: 1 - DNA, 89 bp, synthetic construct
  misc_feature 1..89: note = biotinylated DNA guide
  source 1..89: mol_type = other DNA
ggctaagacagatgctctttgccaacaggccacagaattcctacactaaaagtcgtactgaactatacaacctactacctcatcgcact

SEQ ID NO: 2 - RNA, 22 nt, synthetic construct
  misc_feature 1..22: note = non-coding RNA
  source 1..22: mol_type = other RNA
tgaggtagtaggttgtatagtt

SEQ ID NO: 3 - DNA, 14 bp, synthetic construct
  misc_feature 1..14: note = PCR amplification primer
  source 1..14: mol_type = other DNA
tacgagagatgcga

SEQ ID NO: 4 - DNA, 17 bp, synthetic construct
  misc_feature 1..17: note = PCR amplification primer
  source 1..17: mol_type = other DNA
ggctaagacagatgctc